(19) United States
(12) Patent Application Publication
(10) Pub. No.: US 2024/0399045 A1
(43) Pub. Date: Dec. 5, 2024

(54) EXTRA-LUMEN ADSORPTION OF VIRAL PATHOGENS FROM BLOOD

(71) Applicant: Sigyn Therapeutics, Inc., San Diego, CA (US)

(72) Inventor: James A. Joyce, San Diego, CA (US)

(21) Appl. No.: 18/802,722

(22) Filed: Aug. 13, 2024

Related U.S. Application Data
(63) Continuation of application No. 17/724,845, filed on Apr. 20, 2022, now abandoned.
(60) Provisional application No. 63/177,520, filed on Apr. 21, 2021.

(51) Int. Cl. A61M 1/36; A61M 1/34

(52) U.S. Cl. CPC A61M 1/3679; A61M 1/342; A61M 1/3659; A61M 2202/206; A61M 2205/7509

(57) ABSTRACT

The device described herein converges the plasma separation function of a hollow-fiber plasmapheresis device with a formulation or cocktail of two or more adsorbent components housed in the extra-lumen space (outside the fiber walls, yet inside the outer shell of the plasmapheresis device) to optimize the adsorption of viral pathogens, shed viral proteins and viral exosomes (collectively known as the Viral Targets) in a low-shear force environment without interacting with blood cells.

### RELATED APPLICATIONS

This application is a continuation of and claims priority to U.S. application Ser. No. 17/724,845, filed on Apr. 20, 2022, which claims priority to U.S. Provisional Application Ser. No. 63/177,520, entitled EXTRA-LUMEN ADSORPTION OF VIRAL PATHOGENS FROM BLOOD and filed on Apr. 21, 2021, the contents of each are hereby incorporated by reference in their entirety.

### FIELD OF THE INVENTION

The present invention relates to devices, systems and methods to reduce the presence of viral pathogens and viral particles from blood and blood plasma.

### BACKGROUND

Life-threatening inflammatory conditions, including sepsis are induced by a wide range of infectious and non-infectious conditions that precipitate Cytokine Storm Syndrome. Cytokine Storm Syndrome is an excessive response of the immune system that is induced by infectious and non-infectious conditions. A hallmark indicator of Cytokine Storm Syndrome is the excessive or uncontrolled release of pro-inflammatory cytokines into the bloodstream, which can lead to multiple organ failure and cause death.

The annual market opportunity to address Cytokine Storm related indications exceeds $20 billion and includes sepsis, the most common cause of hospital deaths worldwide. Virus-induced Cytokine Storm Syndrome is a leading cause of death resulting from severe viral infections, including SARS-CoV-2 (COVID-19). Bacterial and viral infections are among the most common sources of life-threatening inflammatory conditions precipitated by cytokine storm syndrome.

There remains a need for a medical device and method of treatment to address inflammatory conditions induced by viral infections.

It should be noted that this Background is not intended to be an aid in determining the scope of the claimed subject matter nor be viewed as limiting the claimed subject matter to implementations that solve any or all of the disadvantages or problems presented above. The discussion of any technology, documents, or references in this Background section should not be interpreted as an admission that the material described is prior art to any of the subject matter claimed herein.

### SUMMARY

Disclosed is a broad spectrum method for treating a viral infection in an individual in need thereof. The method may include providing an extracorporeal adsorptive viral target removal device. Advantageously, the device includes a housing, a hollow fiber plasma filter, and an adsorbent positioned inside the housing and outside the fiber in the extra lumen space. The hollow fiber plasma can have pores sized between about 200-2000 Angstroms.

The method further may include filtering the plasma of an individual through the adsorptive toxin removal device such that the filtering causes a viral target to pass through the pores. The viral target, made up of a viral pathogen, viral protein, viral exosome, and/or particles thereof, is contacted with the adsorbent such that the viral target is bound to the adsorbent. The viral target is then captured in the adsorbent.

In some aspects, the capture of the viral target prevents the viral target from re-entering blood circulation.

The adsorbent may optionally be an activated carbon, non-ionic exchange resin or an ion-exchange resin. The activated carbon may be coated coconut shell granule, uncoated coconut shell granule, or synthetic charcoal. The absorbent can be an ion exchange resin or a non-ionic exchange resin. For the non-ionic exchange resin, it may be a non-ionic aliphatic ester resin, a non-ionic polystyrene divinyl benzene resin, or combinations thereof.

Advantageously, the non-ionic aliphatic ester resins may have an average surface area of approximately 500 m2/g, an average pore size of approximately 300-600 Angstroms, and a mean particle diameter of 560 microns. The non-ionic polystyrene divinyl benzene resins may have an average surface area of approximately 700 m2/g, and an average pore size of 300 Angstroms, and a mean particle diameter from approximately 35 microns to approximately 120 microns. The non-ionic polystyrene divinyl benzene resins may have an average surface area of approximately 600 m2/g, an average pore size of 100-400 Angstroms, and a mean particle diameter from approximately 300 microns to approximately 500 microns.

The activated carbon may have a pore size distribution of a Micropore region of less than 100 Angstroms, a Mesopore region of between 100 and 1,000 Angstroms, and a Macropore region of greater than 1,000 Angstroms.

The viral target may be a bloodborne virus. Suitable bloodborne viruses may include, without limitation HIV, coronavirus, dengue virus, West Nile virus, rubella, measles, cytomegalovirus, Epstein-Barr virus, hepatitis B virus, hepatitis C virus, hepatitis E virus, varicella-zoster virus, chikungunya virus, zika virus, or human T-lymphotropic virus. A bloodborne virus may further include an arenavirus, flavivirus, nairovirus, hantavirus, or phenuivirus.

In another aspect, the method disclosed herein may further include the co- administration of an antiviral agent. The antiviral agent may include an immunostimulator, immunomodulator, a nucleoside antiviral agent, a nucleotide antiviral agent, a protease inhibitor, inosine 5′-monophosphate dehydrogenase (IMPDH) inhibitor, a viral entry inhibitor, a viral maturation inhibitor, a viral uncoating inhibitor, an integrase inhibitor, viral enzyme inhibitor, an anti-sense molecule, a ribozyme antiviral agent, a nanoviricide, interferon, antibody or combinations thereof. The antiviral agent may be administered before, substantially contemporaneously with hemopurification, or after hemopurification.

A kit for treating a viral infection in an individual is likewise disclosed. The kit may include a broad spectrum extracorporeal blood purification device. The device may have a housing and a hollow fiber filter disposed within the housing. Advantageously, the filter includes pores sized and dimensioned to permit passage of viral pathogens and viral particles. The viral pathogens and viral particles may have a diameter between about 20 nm and 200 nm. The device may also include at least one adsorption component positioned inside the housing and outside the hollow fiber in an extra-lumen space.

The kit may optionally include an accessory such as a blood access catheter, a blood tubing set, a blood connector, and combinations thereof. In still another aspect, the kit may include an antiviral agent. The hollow fiber filter of the kit may have an average pore size of 2000 Angstroms. The adsorption component may be activated carbon, non- ionic exchange resin, or an ion exchange resin.

A device configured to perform extracorporeal removal of viral targets from a fluid of a subject is provided. The device may include a housing; a hollow fiber plasma filter having pores sized between 200-2000 Angstroms, and an adsorbent positioned inside the housing and outside the fiber in an extra lumen space.

Advantageously, the device is configured to filter a fluid such as blood or plasma through the device such that the filtering causes a viral target to pass through the pores; wherein the viral target contacts the adsorbent and is bound to the adsorbent; and wherein the viral target is captured in the adsorbent.

It is understood that various configurations of the subject technology will become apparent to those skilled in the art from the disclosure, wherein various configurations of the subject technology are shown and described by way of illustration. As will be realized, the subject technology is capable of other and different configurations and its several details are capable of modification in various other respects, all without departing from the scope of the subject technology. Accordingly, the summary, tables, and detailed description are to be regarded as illustrative in nature and not as restrictive.

### DETAILED DESCRIPTION

The following description and examples illustrate some exemplary implementations, embodiments, and arrangements of the disclosed invention in detail. Those of skill in the art will recognize that there are numerous variations and modifications of this invention that are encompassed by its scope. Accordingly, the description of a certain example embodiment should not be deemed to limit the scope of the present invention.

Implementations of the technology described herein are directed generally to an extracorporeal blood purification device configured to adsorb viral pathogens in the extra-lumen space of said device.

The device described herein converges the plasma separation function of a hollow-fiber plasmapheresis device with a formulation or cocktail of adsorbent components housed in the extra-lumen space (outside the fiber walls, yet inside the outer shell of the plasmapheresis device) to optimize the adsorption of viral pathogens, shed viral proteins and viral exosomes (collectively known as the “Viral Targets”) in a low-shear force environment without interacting with blood cells.

As blood flows into the described device, plasma components along with Viral Targets transport through the porous hollow-fiber walls as a result of the blood side pressure that is established by the rate of blood flow within the hollow-fibers. To allow Viral Targets to transport into the extra-lumen space, the fiber wall pores may range up to 500 nanometers in size. To create sufficient pressure, the flow rate of blood circulated into the plasmapheresis device equals or exceeds 70% of the maximum flow rate guidance for the plasmapheresis device.

As the hollow fiber bundle creates a resistance to the flow of blood, a pressure drop is created along the length of the device such that the blood-side pressure is higher at the blood inlet and lower at the blood outlet. As a result, Viral Targets, plasma and plasma components flow away from the blood along the proximal first half of the fiber bundle length and into the extra-lumen space to interact with two or more adsorbent components. This interaction may be accomplished via sequestration of the virus and/or by binding the virus and viral particles to one or more adsorbent components. In the distal back half of the fiber bundle length, the pressure gradient is reversed, which causes the plasma to flow backward through the fiber walls where it is recombined with cellular blood components with a reduced level of Viral Targets.

An extracorporeal device for the broad-spectrum reduction of Viral Particles in blood and/or plasma is provided. As used herein, the extracorporeal device can be a plasma separation device. In a preferred embodiment, the extracorporeal device is an adsorptive viral particle removal device, wherein blood or plasma is filtered through the device and viral particles which can cause a pro-inflammatory response have diameters less than about 200 nm can pass through pores and be bound, captured, and/or adsorbed by adsorbents in an extra-lumen space. In some embodiments, the viral particle size has a diameter between about 20 nm and 200 nm.

In some embodiments, the device comprises a cartridge housing which can be transparent so as to reveal the internal components of the device. It will be appreciated, however, that the housing may be transparent, translucent, or opaque. Disposed within the housing is a hollow fiber filter comprised of a plurality of hollow fibers having fiber walls and a plurality of pores. The pores are sized and configured to allow Viral Targets in blood or plasma as small as 0.5 nanometers and as large as 200 nanometers to pass through the walls of the hollow fibers. Viral particles with diameters less than 0.20 microns can thus pass through said plurality of pores into an extra-lumen space. By contrast, agents and blood components having diameters greater than about 0.20 microns are blocked by the fiber walls and cannot enter the extra-lumen space. The device further includes an inlet port for receiving unfiltered blood or plasma and an outlet port, wherein filtered blood or plasma exits the device for reintroduction into the circulatory system of the individual/patient.

Notably, the extra-lumen space is populated with an adsorption component. An adsorption component, as used herein, refers to a substance which binds, captures, sequesters, or otherwise adsorbs circulating viral particles which act as inflammatory agents.

The adsorption component can be activated carbon, non-ionic exchange resins, ion exchange resins, or combinations thereof. The activated carbon can include coated coconut shell granule, uncoated coconut shell granule, and/or synthetic charcoal. The activated carbon may have a pore size distribution of a micropore region of less than 100 Angstroms, a mesopore region of between about 100 and 1,000 Angstroms, and a macropore region of greater than 1,000 Angstroms. The non-ionic exchange resin can include non-ionic aliphatic ester resins, non-ionic polystyrene divinyl benzene resins, or any other suitable non-biologic adsorptive resin.

In one aspect, the non-ionic aliphatic ester resin has an average surface area of approximately 500 m2/g, an average pore size of between about 300-600 Angstroms, and a mean particle diameter of about 560 microns. In another aspect, the non-ionic polystyrene divinyl benzene resin has an average surface area of approximately 700 m2/g, an average pore size of about 300 Angstroms, and a mean particle diameter from approximately 35 microns to approximately 120 microns. In still another embodiment, the non-ionic polystyrene divinyl benzene resin has an average surface area of approximately 600 m2/g, an average pore size of 100-400 Angstroms, and a mean particle diameter of between about 300 microns to about 500 microns.

Adsorption component can be applied to carriers which include, but are not limited to, coated or otherwise treated Alginate-Based Hydrogel Beads, perlite beads, Bio-Beads SM-2 Resin, and Bio-Beads S-X beads, or any suitable carrier as will be appreciated by a person of ordinary skill in the art.

The device disclosed herein is well-suited for the broad-spectrum reduction of Viral Targets in the blood. As used herein, Viral Targets refers to viruses and viral particles found in the bloodstream. In a preferred embodiment, the Viral Targets have a diameter of between about 20 nanometers to between about 300 nanometers. In another embodiment, a Viral Target has a diameter of less than 200 nm. The Viral Target can include genetic material (DNA or RNA), a protein coat that protects the genes, and, in some embodiments, an envelope of lipids that surrounds the protein coat. The infectious Viral Target can include spike proteins, which allow for the Viral Target to be sequestered by the adsorbent components of the disclosed device.

The Viral Target can be any virus or viral particle which is bloodborne. A non-exhaustive list of Viral Targets can include, for example, dengue virus, West Nile virus, rubella, measles, cytomegalovirus, Epstein-Barr virus, lentivirus such as HIV, hepatitis B virus, hepatitis C virus, hepatitis E virus, poliovirus, yellow fever virus, varicella-zoster virus, chikungunya virus, zika virus, herpes simplex virus, filoviridae virus, papillomaviruses, parvoviruses, arenavirus, flavivirus, nairovirus, phenuivirus, polyomavirus, adenovirus, coronavirus such as SARS-COV-2, Japanese encephalitis virus, ebola, Marburg virus, Rift Valley fever virus, alkhurma hemorrhagic fever virus, chapare hemorrhagic fever virus, Crimean-Congo hemorrhagic fever virus, hantavirus, Lassa, Marburg, and human T-lymphotropic virus.

Disclosed herein are extracorporeal methods of reducing viral loads to individuals infected with a virus. The method includes providing an adsorptive toxin removal device as described above, having a housing, a hollow fiber plasma filter having a plurality of pores sized between about 200-2000 Angstroms, and a plurality of adsorbents positioned inside the housing and outside the hollow fiber filter. Plasma is filtered through the adsorptive toxin removal device such that Viral Targets having a diameter of less than 0.6 microns can pass through the pores of the hollow fiber filter and enter the extra-lumen space. The Viral Targets are exposed to the plurality of adsorbents int eh extra-lumen space such that the Viral Targets are caused to be bound, captured, sequestered, and/or adsorbed by the adsorbents, thereby reducing the amount of viral load in an individual's plasma.

A method of employing adsorbents in a hollow fiber filtration device to remove pathogenic Viral Targets from infected blood or plasma in an extracorporeal setting is provided. The method can include obtaining blood or plasma from the individual, passing the blood or plasma through a porous hollow fiber filter disposed within a housing. The filter includes a plurality of pores sized and dimensioned to allow passage of Viral Targets. The system further includes an adsorption component positioned inside the housing and outside the hollow fiber in the extra-lumen space. The Viral Targets are bound, sequestered, immobilized, or otherwise captured by the adsorbent material. Pass-through blood or plasma is collected, and reinfusing the pass-through blood or plasma into the individual.

A methodology to reduce the systemic presence of Viral Targets is provided and is initiated through access to a patient's circulatory system. Access to the circulatory system can be obtained from arterial access or venous access. In one embodiment, access is obtained through the insertion of a central venous catheter into a patient. In a preferred embodiment, the catheter is a dual lumen catheter. Prior to initiation, a primary solution, which may include a saline or albumin solution is advantageously circulated throughout the device to improve hemocompatibility. Optionally, anticoagulant agents may be administered.

Once the device has been primed and access to the circulatory system established, the reduction or depletion of inflammatory particles from the blood or plasma occurs as an individual's blood or plasma passes through the extracorporeal device. The device is configured to connect through the extracorporeal lines of the catheter to the patient's circulatory system. A pump facilitates flow from the patient's circulatory system and through the extracorporeal device. The pump can be any approved device suitable for facilitating the extracorporeal filtration of blood and/or plasma. Exemplary pumps include dialysis pumps and CRRT machines.

The device includes walls of porous hollow-fiber membranes, wherein a formulation of adsorbent components are resident outside of the membrane walls and within the extra-lumen space between the outer shell of the cartridge and the hollow-fibers. The adsorbent components are formulated to bind, capture or adsorb a broad-spectrum of Viral Targets that pass through the fiber walls to interact with the adsorbent components. As will be appreciated by a person of skill in the art, the blood or plasma is circulated to flow at rates sufficient to create pressure to cause plasma and Viral Targets to flow through the fiber walls, but not at rates that would cause hemolysis. As blood or plasma is filtered through the device, the population of viruses and viral particles is captured and reduced from the entire bloodstream, which is continuously infused back into the patient at rates equal to its removal during treatment.

Aspects of the invention are based upon the surprising discovery of a system and method for removing a broad spectrum of Viral Targets using a single extracorporeal device. The removal of Viral Targets in a single device without harming critical blood components creates considerable therapeutic benefits.

A method of treating an individual with virally infected blood is likewise provided. The treatment protocol can include providing an adsorptive Viral Target extracorporeal removal device having hollow fibers and adsorbent components in the extra-lumen space. The device includes a housing, a hollow fiber plasma filter having a plurality of pores sized between about 200-2000 Angstroms, and a plurality of adsorbents positioned inside the housing and outside the hollow fiber filter. Plasma is filtered through the adsorptive toxin removal device such that Viral Targets having a diameter less than 0.20 microns can pass through the pores of the hollow fiber filter and enter the extra-lumen space. The Viral Targets are exposed to the plurality of adsorbents in the extra-lumen space such that the Viral Targets are caused to be bound, captured, and/or adsorbed by the adsorbents, thereby reducing the amount of Viral Targets in an individual's plasma.

The method can also include combination therapy with conventional anti-viral agents administered before, after, or substantially contemporaneously with blood filtration. Accordingly, embodiments of the present disclosure enhance the efficacy of an antiviral therapy by combining the antiviral therapy with a method that physically removes virus and/or viral particles to reduce viral load. The multi-function blood purification technology disclosed herein can improve the effectiveness and extend the benefit of an antiviral therapy compared to administering of either the extracorporeal blood purification treatment or the course of antiviral therapy alone.

The combination of the two therapies can have a number of benefits. When the blood purification treatment is administered less than continuously, (e.g. 4 to 8 hours a day, 1 to 7 times a week) there can be a rebound in the viral load between treatments. By combining hemodialysis with antiviral therapy, the rebound in viral load between blood purification treatments is reduced, resulting in a lower average viral load for the subject during the period of blood purification treatment plus antiviral therapy as compared to blood purification treatment alone. This can be seen as lower viral loads prior to the initiation of each individual blood purification treatment during the course of therapy, or in the average viral load during the blood purification therapy.

The combination of the two therapies can also result in the absence, or lessening of viral load rebound following the cessation of blood purification therapy. Extracorporeal filtration of infected blood through hollow fibers and adsorbents therapy alone, or antiviral therapy alone, can achieve significant reductions in viral load. However, once either therapy is stopped, the viral load can begin to increase. This rebound in viral load can also be seen in patients that continue viral therapy as the virus adapts and becomes resistant to the antiviral being used. The combined therapy can reduce the level of rebound, preferably keeping it below a clinically or therapeutically relevant level. Alternatively, or in addition, the combination therapy can lengthen the amount of time before any rebound in viral load is seen.

In any of the embodiments, the use of a blood purification device and antiviral therapy improves the effectiveness of the method or treatment compared to either the disclosed device or antiviral therapy alone. In a preferred embodiment, the improvement is additive, more preferably greater than additive, e.g. synergistic.

In some embodiments, a kit useful for practicing the methods described herein is provided. Such a kit generally comprises an extracorporeal device with hollow fibers and an adsorbent in the extra-lumen space as described herein. In some aspects of the invention, the kit can include blood access catheters, blood tubing sets and/or connectors. It may optionally include at least one antiviral agent. In some embodiments, the antiviral agent can be any of the antiviral agents disclosed herein. In some embodiments, the kit contains instructions for administering the antiviral agent and/or using the blood purification device. The kit or any component of the kit can be presented in a commercially packaged form. The kit can be packaged in combination with one or more containers, devices, or necessary reagents and written or electronic instructions for the performance of the methods described herein. In some embodiments, the kit contains no less than one blood purification device and a daily dose of the antiviral agent. In some embodiments, the kit contains no less than one blood purification device and the antiviral agent sufficient for 3 days of treatment. In some embodiments, the kit contains no less than one syringe.

In some embodiments, the antiviral agent is selected from the group consisting of immunostimulators, immunomodulators, nucleoside antiviral agents, nucleotide antiviral agents, protease inhibitors, inosine 5′-monophosphate dehydrogenase (IMPDH) inhibitors, viral entry inhibitors, viral maturation inhibitors, viral uncoating inhibitors, integrase inhibitors, viral enzyme inhibitors, anti-sense molecules, ribozyme antiviral agents, nanoviricides, interferons and antibodies. In some embodiments, the antiviral agent is selected from the group consisting of remdesivir, paxlovid, molnupiravir, nitazoxanide, bictegravir, nirmatrelvir, ritonavir, sotrovimab, bebtelovimab, tocilizumab, baricitinib, emtricitabine, tenofovir alafenamide, amantadine, rimantadine, pleconaril, acyclovir, zidovudine, lamivudine, fomivirsen, zanamivir (Relenza) and oseltamivir (Tamiflu). In another embodiment, the antiviral agent is convalescent plasma. The antiviral agent can be a monoclonal antibody medication such as bamlanivimab and etesevimab.

The antiviral agent can be stored in single-use vials or packages, or multiple- use vials or packages. The antiviral agent can be administered to the patient through: (a) oral pathways, which includes administration in capsule, tablet, granule, spray, syrup, or other such forms; (b) administration through non-oral pathways such as rectal, vaginal, intraurethral, intraocular, intranasal, or intraarticular, which includes administration as an aqueous suspension, an oily preparation or the like or as a drip, spray, suppository, salve, ointment or the like; (c) administration via injection, subcutaneously, intraperitoneally, intravenously, intramuscularly, intradermally, intraorbitally, intracapsularly, intraspinally, intrasternally, or the like, including infusion pump delivery; (d) administration locally such as by injection directly in the renal or cardiac area, e.g., by depot implantation; as well as (e) administration topically; as deemed appropriate by those of skill in the art for bringing the antiviral agent into contact with living tissue.

The term “viral load reduction rate” is defined as a rate at which the viral load is reduced, and refers to the amount of time required for an enhanced antiviral therapy, or an antiviral therapy, or a blood purification therapy to clear, or remove, a specific amount of viruses or viral particles from blood of a patient. For example, a system or treatment capable of reducing a viral load of 10×109 copies by half (that is, to 5×109 copies) in 1 hour has a viral load reduction rate of 5×109 copies/hour (or 50% per hour), and a T½ or T50% value of 1 hour. A system capable of reducing a viral load of 10x109 copies by 90% (that is, to 1×109 copies) in 1 hour has a viral load reduction rate of 9×109 copies/hour (or 90% per hour), and a T90% value of 1 hour.

In some embodiments, the reduction in viral load is measured by comparing the viral load of the patient immediately before the start of a session with an extracorporeal blood purification system having hollow fibers and adsorbents and the viral load of the patient immediately after the completion of that session. In some embodiments, the reduction in viral load is measured for every session during the course of treatment or during the course of an enhanced antiviral therapy. In some embodiments, the reduction in viral load is measured every hour, every 4 hours, every 8 hours, every 12 hours, everyday, or every other day during the course of an antiviral therapy or during the course of an enhanced antiviral therapy. In some embodiments, the reduction in viral load follows a log linear clearance according to the formula: C=Co e-kt/V, where C=virus concentration, k=constant of viral load reduction (=In2/t½;); t½=time to reduce the viral load by 50%; and V=blood volume of the patient. In some embodiments, the formula assumes a constant blood flow rate.

In some embodiments, the viral load reduction rate is, is about, is less than, is less than about, is more than, is more than about, 1×104 copies/hour, 5×104 copies/hour, 1×105 copies/hour, 5×105 copies/hour, 1×106 copies/hour, 5×106 copies/hour, 1×107 copies/hour, 5×107 copies/hour, 1×108 copies/hour, 5×108 copies/hour, 1×109 copies/hour, 5×109 copies/hour, 1×1010 copies/hour, 5×1010 copies/hour, 1×1011 copies/hour, 5×1011 copies/hour, 1×1012 copies/hour, or 5×1012 copies/hour, 1×104 copies/day, 5×104 copies/day, 1×105 copies/day, 5×105 copies/day, 1×106 copies/day, 5×106 copies/day, 1×107 copies/day, 5×107 copies/day, 1×108 copies/day, 5×108 copies/day, 1×109 copies/day, 5×109 copies/day, 1×1010 copies/day, 5×1010 copies/day, 1×1011 copies/day, 5×1011 copies/day, 1×1012 copies/day, or 5×1012 copies/day or a range defined by any two of these values. In some embodiments, the viral load reduction rate is, is about, is less than, is less than about, is more than, is more than about, 0.1% per hour, 0.25% per hour, 0.5% per hour, 1% per hour, 2.5% per hour, 5% per hour, 10% per hour, 15% per hour, 20% per hour, 25% per hour, 30% per hour, 40% per hour, 50% per hour, 60% per hour, 70% per hour, 80% per hour, or 90% per hour, or 0.1% per day, 0.25% per day, 0.5% per day, 1% per day, 2.5% per day, 5% per day, 10% per day, 15% per day, 20% per day, 25% per day, 30% per day, 40% per day, 50% per day, 60% per day, 70% per day, 80% per day, or 90% per day, or a range defined by any of these two values. In some embodiments, continuous reduction in viral load is performed with slower reduction rates (for example, 5% per hour or less), for up to 24 hours per day over one, two, three or more days or weeks. In some embodiments, T½ or T50% is, is about, is less than, is less than about, is more than, is more than about, 15, 30, or 45 minutes, or 1, 2, 3, 4, 5, 6, 7, 8, 9, 10, 11, 12, 13, 14, 15, 16, 17, 18, 19, 20, 21, 22, 23, or 24 hours, or a range defined by any two of these values. In some embodiments, T90% is, is about, is less than, is less than about, is more than, is more than about, 1, 2, 3, 4, 5, 6, 7, 8, 9, 10, 11, 12, 13, 14, 15, 16, 17, or 18 hours, or a range defined by any two of these values.

The efficacy of the device and method for capturing viral particles has been evaluated in in vitro studies and presented in the below Examples. To summarize, liposome, adenovirus, and lentivirus in vitro studies were performed to assess reduction in viral load when passed through the disclosed device. The term “viral load” as used herein for the purpose of specification and claims refers to the amount of viral particles or toxic fragments thereof in a biological fluid, such as blood or plasma. Viral load is accordingly related to the number of virus particles in the body. Viral load can therefore be a measure of any of a variety of indicators of the presence of a virus, such as viral copy number per unit of blood or plasma or units of viral proteins or fragments thereof per unit of blood or plasma. The study methodologies are set forth in detail below.

### EXAMPLES

Preliminary testing with the disclosed dialysis-like blood purification device, consisting of 3 sorbent components contained within the extralumenal space of a plasmapheresis cartridge was performed, with promising results. In Round 1 testing miniaturized sorbent devices (ST100) were fabricated and tested with human plasma doped with 3 cytokines and endotoxin, and demonstrated removal over a short treatment duration of 4 hours. The filtration of virally infected plasma through a miniaturized sorbent device resulted in the depletion of viral pathogens and inflammatory cargos that are transported by microvesicular particles in the bloodstream.

In Round 2 of testing, a sorbent slurry was used in pilot studies to assess the potential of the sorbent system to remove larger molecular weight targets such as microvesicular particles and viruses. Fluoresecent liposomes (˜100 nm) were chosen as a model analog for microvesicular particles. There is ample literature supporting the use of liposomes with lipid and cholesterol constituents as a model system for microvesicular particles (1).

Two types of viral systems were assessed: adenovirus and lentivirus, with adenovirus (90-100 nm) being a DNA-based virus involved in human disease and lentivirus (80-120 nm including envelope), an RNA-based retrovirus that is a prime model for common infectious viruses implicated in human disease including mosquito-borne, pandemic/zoonotic and biothreat viruses.

### Methods and Materials

Sorbent slurry preparation: During Round 1 testing, a specified sorbent admixture (Table 1) was prepared by wetting with 70% ethanol, and collection using a 10- micron filter and Buchner funnel setup with the aid of vacuum.

TABLE 1
Sorbent admixture used for rocker studies

| Sorbent Component | Amount (g) |
|---|---|
| Activated Carbon | 70.7 |
| Amberlite | 28.3 |
| Amberchrom | 28.3 |
| Total | 126.3 |

Sorbents were suspended in ˜250 mL of solvent, which resulted in a total apparent volume of ˜380 mL. Approximately 200 mL of the sorbent slurry stock solution remained after Round 1 testing. Some of this stock was used in Round 2 testing. Well-mixed sorbent slurry was poured onto filter paper, and excess solvent was removed via vacuum setup. Sorbent was weighed out (1.00 ±0.05 g or 2.00 ±0.10 g as applicable) and added to test tubes.

For Lentivirus testing, the ethanol-wetted, filtered sorbent preparation while on filter paper, went through a final preparation step of rinsing with the specified Normosol-R based priming solution, without heparin due to potential viral inhibition. The sorbent slurry utilized should have had at least partial replacement of ethanol in sorbent pores with Normosol.

Rocker study format: Test tubes filled with sorbent and target solutions (liposomes, adenovirus, or lentivirus, each in separate studies) were rocked on a nutator rocker (multidirectional tilting rocker) at room temperature for 2 hrs. Test tubes were centrifuged for a minimum of 1,500×g for an at least 10 minutes, to allow for the separation of plasma/target mixture from sorbent. In most cases, even after 10 minutes of centrifugation, supernatant was still a bit cloudy with some fine carbon particles, but otherwise appeared free of sorbent constituents. Full removal of carbon from supernatant was not attained with subsequent spins. Carbon-free zones of supernatant were sampled as much as possible as aliquots for fluorescent, absorbance or ELISA analysis on an M5 Spectrophotometer.

Liposome Preparation: Fluorescent liposomes (Formumax F60103F-R, 100 nm, Rhodamine labeled) were prepared at an initial concentration of ˜0.5 mM, in filtered human plasma. Of this stock, 2 mL of human plasma with liposomes was mixed with 2 g of the Sorbent slurry (prepared per ratio and filtered using a 0.2 micron filter) in a 5 ml capacity test tube. A small aliquot of initial liposome stock solution in plasma was also retained to confirm initial fluorescence.

M5 Spectrophotometer analysis of liposomes: A Rhodamine-labeled liposome standard curve was generated for 100 μL stock liposome samples (halving dilution series starting at 5 mM) loaded into a blackout plate, with fluorescence excitation at 560 nm, and emission at 580 nm, with an auto-cutoff value of 570 nm. Initial liposome/plasma solution was shown to have a starting concentration of 0.572 mM. Minimum detection threshold for this assay was ˜0.019 mM.

Adenovirus Preparation: Adenovirus (Ad5 E3 E15, vector: Ad CMV pLpA.dIE3 #1) from University of Michigan Vector Core (U-M VC) was received as a 0.25 mL vial with 4.00×1012 viral particles (VP)/mL, with a corresponding infection titer of 1.09×109. A little over 50 μL was used to establish a standard ladder (in PBS), and the remaining ˜200 μL was prepared as a 1 mL solution in PBS, as it was found that plasma interfered with VP measurement via absorbance at 260 nm. The estimated starting concentration of adenovirus VP solution was ˜8×1011. Of the starting solution, 50 μL was sampled for exact quantification of physical titer.

M5 Spectrophotometer analysis of adenovirus: Adenovirus physical titer of viral particles (VP) was assessed with absorbance of 260 nm. Note that the U-M VC utilizes a NanoDrop reader, and has previously found that an OD260=1, is equivalent to 1×1012 VP/mL. Initial standard ladder construction suggested that the dynamic range of detection would allow for a minimum detectable value of 6.25×1010 VP/mL, or a maximum measurable reduction of ˜92% from the estimated initial solution.

VSVg pseudotyped Lentivirus Preparation: Lentivirus (Lenti-GF1-CMV-VSVG) from University of Michigan Vector Core (U-M VC) was supplied as a 10 mL preparation, with transduction efficiency of 85% and a measured functional titer of 2.66×107 TU/mL. The larger stock supply of 10 mL was thawed, and 1 mL aliquots of stock were prepared to minimize freeze/thaw cycling. Of a 1 mL aliquot, 200 μL of the stock was utilized in 800 μL of human plasma (to closely match the same conditions utilized for Lentivirus-SAR-CoV-2), for a total starting solution volume of 1 mL. Of the starting solution, 50 μL was sampled for exact quantification of physical titer.

M5 Spectrophotometer analysis of Lentivirus-VSVg: To establish a physical titer, Cell BioLabs p24 ELISA assay was utilized with a standard ladder of 100 ng/ml to 1.56 ng/ml. According to the Cell BioLabs kit specifications, there are approximately 2,000 molecules of p24 per lentivirus particle, which generally corresponds to detection range of 8-80 ng/mL=106 TU/mL=108-9 VP/mL (2-4). For this viral stock, the U-M VC established a titer of 2.66x107 TU/mL. Based on the amount used, the initial solution for the 2 hr rocker study was an estimated 5.32×106 TU/mL. Initial solution was diluted 1:2, and final samples were run undiluted.

SAR-CoV-2 pseudotyped Lentivirus Preparation: Lentivirus (Lenti-GF1-SARsCoV2S19AA) from University of Michigan Vector Core (U-M VC) was prepared from an ˜0.2 mL, 2.13 x107 TU/mL stock. The full 200uL stock was utilized in 800 μL of human plasma for a total starting solution volume of 1 mL. Of the starting solution, 50 μL was sampled for exact quantification of physical titer.

M5 Spectrophotometer analysis of Lentivirus-SARS-CoV-2: Similar to the VSVg pseudotyped virus, the same Cell BioLabs p24 ELISA assay was utilized with a standard ladder of 100 ng/ml to 1.56 ng/ml. Based on the amount used, the initial solution for the 2 hr rocker study was an estimated 4.26×106 TU/mL. Initial solution was diluted 1:2, and final samples were run undiluted.

### RESULTS

Liposomes: All values were measurable based on the fluorescence standard ladder. Initial liposome/plasma solution was shown to have a starting concentration of 0.572 mM, which dropped to a value of 0.043 mM in 2hrs, suggesting a liposome reduction of 92.5% with sorbent exposure.

Adenovirus: All values were measurable based on the adenovirus standard ladder at absorbance OD=260 nm. The actual initial adenovirus solution was found to be 1.02×1012, which was reduced to 4.22×1011 following 2 h of sorbent exposure, suggesting a reduction of 59.5%.

VSVg pseudotyped Lentivirus: All values were measurable based on the p24 ELISA standard ladder. The initial solution was found to have 131.5 ng p24/mL, which was reduced to 61.0 ng p24/mL following 2h of sorbent exposure, suggesting a reduction of VSVg pseudotyped lentivirus by 53.6%.

SARS-CoV-2 pseudotyped Lentivirus: All values were measurable based on the p24 ELISA standard ladder. The initial solution was found to have 124.7 ng p24/mL, which was reduced to 38.9 ng p24/mL following 2h of sorbent exposure, suggesting a reduction of SARS-CoV-2 pseudotyped lentivirus by 68.8%.

Percent reduction was calculated for each target, and summarized in Table 2:

TABLE 2
Target removal from human plasma by
Sigyn Therapy sorbent admixture

| | | | | SARS- |
|---|---|---|---|---|
| | | Adeno- | VSVg- | CoV-2 |
| Parameter | Liposomes | virus | Lentivirus | Lentivirus |
| Method | Fluorescence | Ab- | p24 | p24 |
| | | sorbance | ELISA | ELISA |
| Initial | 0.572 mM | 1.02 × | 131.5 ng | 124.7 ng |
| Concen- | | 10¹² | p24/mL | p24/mL |
| tration | | VP/mL | | |
| Concen- | 0.043 mM | 4.22 × | 61.0 ng | 38.9 ng |
| tration | | 10¹¹ | p24/mL | p24/mL |
| after 2 h | | VP/mL | | |
| % | 92.5% | 59.5% | 53.6% | 68.8% |
| Reduction | | | | |

### Measurement of Extra-Capillary Space (ECS) of Medica Plasmart (Versatile®-PES Plasmafilter) to Establish Sorbent Filling Protocols for Good Manufacturing Process (GMP) Sigyn Devices

Plasmart 100, 600 and 1000 devices were unpackaged, inspected, dry mass was determined with end caps in place, and ECS (aka extralumenal space) volume was measured by quickly filling with sterile water for injection (SWFI) while keeping the lumen tightly capped (Table 3). SWFI fill volume was tracked closely looking for signs of bubbles or lumen filling. Fluid-filled device mass was measured to precisely determine ECS fill volume requirements.

TABLE 3
Sigyn Therapy plasmapheresis
cartridge core specifications

| | | Membrane | Lumen | ECS |
|---|---|---|---|---|
| | Mass | area | Prime | volume |
| Device | (g) | (m²) | (mL) | (mL) |
| Plasmart 100 | 71.6 | 0.1 | 8.6 | 60 |
| Plasmart 600 | 165.4 | 0.6 | 48.5 | 203 |
| Plasmart 1000 | 250.8 | 1.0 | 78.3 | 307 |

### DISCUSSION

Initial and final concentrations of each target tested suggests target reduction, with liposomes (a model for exosomes ˜100 nm in size) exhibiting a great reduction elicited by this sorbent formulation (>92%) in 2 hours. Viral target reduction was lower, but substantial, for both adenovirus (90-100 nm) and lentivirus (80-120 nm). This wide range of clinically relevant target removal including low molecular weight (LMW) targets (as demonstrated in Round 1 testing with FD&C “Red 3”, AKA: erythrosine, with a MW=880 Da), middle MW including cytokines (generally 10-30 kDa) and endotoxin (generally ˜10 kDa, but can form larger aggregates) and larger viral targets (80-120 nm, equivalent to molecular weights in the MDa range) that appears to be novel.

In blood studies, therapeutic targets in blood smaller than the pores of the plasma filter (˜200 nm) used in the disclosed device will diffuse across the hollow fibers into the ECS compartment. Targets then are subject to bulk flow of plasma in the ECS compartment and interaction with the sorbent constituents for binding/sequestration leading to reduction in circulating viral pathogens.

These studies support a broad set of therapeutic viral targets that may be reduced through the disclosed sorbent system treatment. To date, liposomes have been used as a model system for exosomes, and adenovirus and lentivirus targets have been shown to be reduced in rocker studies. Cytokines and endotoxin removal have been tested in recirculating plasma studies with miniaturized devices. Results from these model systems to translate to target removal by the clinical version of the disclosed device.

### General Interpretive Principles for the Present Disclosure

Various aspects of the novel systems, apparatuses, and methods are described more fully hereinafter with reference to the accompanying drawings. The teachings disclosure may, however, be embodied in many different forms and should not be construed as limited to any specific structure or function presented throughout this disclosure. Rather, these aspects are provided so that this disclosure will be thorough and complete, and will fully convey the scope of the disclosure to those skilled in the art. Based on the teachings herein one skilled in the art should appreciate that the scope of the disclosure is intended to cover any aspect of the novel systems, apparatuses, and methods disclosed herein, whether implemented independently of or combined with any other aspect of the disclosure. For example, a system or an apparatus may be implemented, or a method may be practiced using any one or more of the aspects set forth herein. In addition, the scope of the disclosure is intended to cover such a system, apparatus or method which is practiced using other structure, functionality, or structure and functionality in addition to or other than the various aspects of the disclosure set forth herein. It should be understood that any aspect disclosed herein may be set forth in one or more elements of a claim. Although some benefits and advantages of the preferred aspects are mentioned, the scope of the disclosure is not intended to be limited to particular benefits, uses, or objectives. The detailed description and drawings are merely illustrative of the disclosure rather than limiting, the scope of the disclosure being defined by the appended claims and equivalents thereof.

With respect to the use of plural vs. singular terms herein, those having skill in the art can translate from the plural to the singular and/or from the singular to the plural as is appropriate to the context and/or application. The various singular/plural permutations may be expressly set forth herein for sake of clarity.

When describing an absolute value of a characteristic or property of a thing or act described herein, the terms “substantial,” “substantially,” “essentially,” “approximately,” and/or other terms or phrases of degree may be used without the specific recitation of a numerical range. When applied to a characteristic or property of a thing or act described herein, these terms refer to a range of the characteristic or property that is consistent with providing a desired function associated with that characteristic or property.

In those cases where a single numerical value is given for a characteristic or property, it is intended to be interpreted as at least covering deviations of that value within one significant digit of the numerical value given.

If a numerical value or range of numerical values is provided to define a characteristic or property of a thing or act described herein, whether or not the value or range is qualified with a term of degree, a specific method of measuring the characteristic or property may be defined herein as well. In the event no specific method of measuring the characteristic or property is defined herein, and there are different generally accepted methods of measurement for the characteristic or property, then the measurement method should be interpreted as the method of measurement that would most likely be adopted by one of ordinary skill in the art given the description and context of the characteristic or property. In the further event there is more than one method of measurement that is equally likely to be adopted by one of ordinary skill in the art to measure the characteristic or property, the value or range of values should be interpreted as being met regardless of which method of measurement is chosen.

It will be understood by those within the art that terms used herein, and especially in the appended claims (e.g., bodies of the appended claims) are intended as “open” terms unless specifically indicated otherwise (e.g., the term “including” should be interpreted as “including but not limited to,” the term “having” should be interpreted as “having at least,” the term “includes” should be interpreted as “includes but is not limited to,” etc.).

It will be further understood by those within the art that if a specific number of an introduced claim recitation is intended, such an intent will be explicitly recited in the claim, and in the absence of such recitation no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should typically be interpreted to mean “at least one” or “one or more”); the same holds true for the use of definite articles used to introduce claim recitations. In addition, even if a specific number of an introduced claim recitation is explicitly recited, those skilled in the art will recognize that such recitation should typically be interpreted to mean at least the recited number (e.g., the bare recitation of “two recitations,” without other modifiers, typically means at least two recitations, or two or more recitations).

In those instances where a convention analogous to “at least one of A, B, and C” is used, such a construction would include systems that have A alone, B alone, C alone, A and B together without C, A and C together without B, B and C together without A, as well as A, B, and C together. It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include A without B, B without A, as well as A and B together.”

Various modifications to the implementations described in this disclosure can be readily apparent to those skilled in the art, and generic principles defined herein can be applied to other implementations without departing from the spirit or scope of this disclosure. Thus, the disclosure is not intended to be limited to the implementations shown herein but is to be accorded the widest scope consistent with the claims, the principles and the novel features disclosed herein. The word “exemplary” is used exclusively herein to mean “serving as an example, instance, or illustration.” Any implementation described herein as “exemplary” is not necessarily to be construed as preferred or advantageous over other implementations.

Certain features that are described in this specification in the context of separate implementations also can be implemented in combination in a single implementation. Conversely, various features that are described in the context of a single implementation also can be implemented in multiple implementations separately or in any suitable sub-combination. Moreover, although features can be described above as acting in certain combinations and even initially claimed as such, one or more features from a claimed combination can in some cases be excised from the combination, and the claimed combination can be directed to a sub-combination or variation of a sub-combination.

The methods disclosed herein comprise one or more steps or actions for achieving the described method. The method steps and/or actions may be interchanged with one another without departing from the scope of the claims. In other words, unless a specific order of steps or actions is specified, the order and/or use of specific steps and/or actions may be modified without departing from the scope of the claims.

What is claimed is:

1. A method for treating a viral infection in an individual in need thereof, comprising:
providing an extracorporeal adsorptive viral target removal device, said device having:
a housing;
a hollow fiber plasma filter having a plurality of pores sized between 200-2000 Angstroms and
an adsorbent positioned inside the housing and outside the fiber in an extra lumen space;
filtering the plasma of an individual in need thereof through said adsorptive viral target removal device; wherein said filtering causes a viral target to pass through said pores;
contacting said viral target with said adsorbent; wherein said viral target is bound to said adsorbent; and
capturing said viral target in said adsorbent.

2. The method of claim 1, wherein the capture of said viral target prevents the viral target from reentering circulation.

3. The method of claim 1, wherein said viral target is selected from the group consisting of a viral pathogen, viral protein, viral exosome, viral particle and combinations thereof.

4. The method of claim 1, wherein said adsorbent is selected from the group consisting of activated carbon, non-ionic exchange resin and ion exchange resin.

5. The method of claim 1, wherein said viral target is a bloodborne virus.

6. The method of claim 5, wherein said virus is selected from the group consisting of HIV, dengue virus, West Nile virus, rubella, measles, cytomegalovirus, Epstein-Barr virus, hepatitis B virus, hepatitis C virus, hepatitis E virus, varicella-zoster virus, chikungunya virus, zika virus, and human T-lymphotropic virus.

7. The method of claim 5, wherein said virus is selected from the group consisting of arenavirus, flavivirus, nairovirus, hantavirus, and phenuivirus.

8. The method of claim 5, wherein said virus is a coronavirus.

9. The method of claim 4, wherein said activated carbon is selected from the group consisting of coated coconut shell granule, uncoated coconut shell granule, and synthetic charcoal.

10. The method of claim 4, wherein the adsorbent is at least one ion exchange resin or non-ionic exchange resin.

11. The method of claim 4, wherein said at least one non-ionic exchange resin is a non-ionic aliphatic ester resins, non-ionic polystyrene divinyl benzene resins, or combinations thereof.

12. The method of claim 11, wherein at least one of said non-ionic aliphatic ester resins has an average surface area of approximately 500 m2/g, an average pore size of approximately 300-600 Angstroms, and a mean particle diameter of 560 microns.

13. The method of claim 11, wherein at least one of said non-ionic polystyrene divinyl benzene resins has an average surface area of approximately 700 m2/g, and an average pore size of 300 Angstroms, and a mean particle diameter from approximately 35 microns to approximately 120 microns.

14. The method of claim 11, wherein at least one of said non-ionic polystyrene divinyl benzene resins has an average surface area of approximately 600 m2/g, an average pore size of 100-400 Angstroms, and a mean particle diameter from approximately 300 microns to approximately 500 microns.

15. The method of claim 4, wherein the activated carbon has a pore size distribution of a Micropore region of less than 100 Angstroms, a Mesopore region of between 100 and 1,000 Angstroms, and a Macropore region of greater than 1,000 Angstroms.

16. The method of claim 1, further comprising administering an antiviral agent selected from the group consisting of an immunostimulator, immunomodulator, a nucleoside antiviral agent, a nucleotide antiviral agent, a protease inhibitor, inosine 5′-monophosphate dehydrogenase (IMPDH) inhibitor, a viral entry inhibitor, a viral maturation inhibitor, a viral uncoating inhibitor, an integrase inhibitor, viral enzyme inhibitor, an anti-sense molecule, a ribozyme antiviral agent, a nanoviricide, interferon, antibody and combinations thereof.

17. A kit for treating a viral infection in an individual in need thereof, the kit comprising:
a broad spectrum extracorporeal blood purification device, comprising
a housing;
a hollow fiber filter disposed within said housing, said filter comprising a plurality of pore sized and dimensioned to permit passage of viral targets having a diameter between about 20 nm and 200 nm; and
at least one adsorption component positioned inside the housing and outside the hollow fiber in an extra-lumen space.

18. The kit of claim 17, further comprising an accessory selected from the group consisting of a blood access catheter, a blood tubing set, a blood connector, and combinations thereof.

19. The kit of claim 17, wherein said hollow fiber filter has an average pore size of 2000 Angstroms.

20. The kit of claim 17, wherein said wherein said adsorption component is selected from the group consisting of activated carbon, non-ionic exchange resin and ion exchange resin.
